(19) United States
(12) Patent Application Publication
(10) Pub. No.: US 2022/0370325 A1
(43) Pub. Date: Nov. 24, 2022

(54) COSMECEUTICAL COMPOSITION

(71) Applicant: OHIO STATE INNOVATION FOUNDATION, Columbus, OH (US)

(72) Inventors: Jianjie MA, POWELL, OH (US); Jianjun GUAN, WILDWOOD, MO (US); Haichang LI, DUBLIN, OH (US); Hong NIU, ST. LOUIS, MO (US); Ya GUAN, CLAYTON, MO (US); Tao TAN, COLUMBUS, OH (US)

(73) Assignee: OHIO STATE INNOVATION FOUNDATION, Columbus, OH (US)

(21) Appl. No.: 17/871,412

(22) Filed: Jul. 22, 2022

Related U.S. Application Data
(63) Continuation of application No. PCT/US2021/012749, filed on Jan. 8, 2021.
(60) Provisional application No. 62/964,974, filed on Jan. 23, 2020.

(51) Int. Cl. A61K 8/64; A61K 8/27; A61K 8/04; A61K 8/81; A61Q 19/00; A61Q 7/00

(52) U.S. Cl. CPC A61K 8/64; A61K 8/27; A61K 8/042; A61K 8/8129; A61Q 19/005; A61Q 7/00

(57) ABSTRACT

Cosmeceutical compositions for and methods of improving the appearance of skin and promoting hair growth are provided. Said compositions comprise at least one ROS-scavenging polymer gel, at least MG53, or a combination thereof. A novel boronate ester-based copolymer forms an aqueous hydrogel exhibiting lower viscosity at cooler temperatures and higher viscosity at higher temperatures is provided.

### CROSS-REFERENCE TO EARLIER-FILED APPLICATIONS

The present application claims the benefit of and is a continuation of application No. PCT/US2021/012749 filed Jan. 8, 2021, which claims the benefit of application No. 62/964,974 filed Jan. 23, 2020, the entire disclosures of which are hereby incorporated by reference.

### STATEMENT REGARDING FEDERALLY SPONSORED RESEARCH

The invention was made with government support under RO1 AG056919 awarded by the National Institutes of Health and under R44 GM123887 awarded by the National Institutes of Health. The government has certain rights in the invention.

### FIELD OF THE INVENTION

The present invention concerns cosmeceutical compositions for topical administration to subjects in need thereof. These compositions improve the appearance of skin and promote healthy growth of cells following repeated topical administration. The compositions comprise boronate ester polymer or MG53 or combinations thereof.

### BACKGROUND OF THE INVENTION

Cosmeceutical compositions have been marketed as exhibiting extraordinary clinical benefits, such as reducing wrinkles, removing age spots, etc.; however, many such compositions fail to provide such benefits. Moreover, many such compositions require a complex mixture of active compounds and excipients. For example, such compositions typically require a polymeric matrix in admixture with one or more antioxidant compounds or a polymeric matrix with an antioxidant compound covalently bound to the matrix.

Methods of preparing and/or isolating MG53 are known: U.S. Pat. No. 7,981,866, WO2008/054561, WO2009/073808, US2011/0202033, US2011/0287004, US2011/0287015, US2013/0123340, WO2011/142744, WO2012/061793, U.S. Pat. Nos. 8,420,338, 9,139,630, 9,458,465, 9,494,602, US2014/0024594, WO2012/134478, WO2012/135868, US2015/0110778, WO2013/036610, US2012/0213737, WO2016/109638.

Polymers containing boronate esters are known: US200802800856, US20060276435, US20130302529, Ryu et al. (“Phenylboronic acid-polymers for biomedical applications” in Curr. Med. Chem. (2018), doi: 10.2174/0929867325666181008144436), and Jager et al. (“Fluorescent boronate-based polymer nanoparticles with reactive oxygen species (ROS)-triggered cargo release for drug-delivery applications” in Nanoscale (2016) 8(13), 6958-6963).

It would be an advancement in the art to provide a cosmeceutical composition that does not require a complex mixture of ingredients and still provides improved clinical benefit.

### SUMMARY OF THE INVENTION

The present invention seeks to provide compositions for improving the appearance of skin and promoting healthy growth of tissue and hair follicles following repeated topical administration.

An aspect of the invention provides a cosmeceutical composition comprising at least one ROS-scavenging polymer, otherwise referred to herein as an antioxidant polymer and ROS meaning “reactive oxygen species”. Accordingly, the ROS-scavenging polymer of the invention is able to scavenge such oxygen intermediates so that they do not damage tissue. In some embodiments, the ROS-scavenging polymer comprises a boronate ester-based copolymer. The copolymer reacts with the ROS to sequester, neutralize or eliminate the ROS.

Unlike other polymeric materials that exhibit increased viscosity at lower temperatures and decreased viscosity of higher temperature, an aqueous hydrogel of the ROS-scavenging polymer exhibits a lower viscosity at about 4° C. and a higher viscosity (solid, semi-solid, paste, gum, gel, or flexible (pliable) solid) at about 37° C. In some embodiments, the ROS-scavenging polymer-containing hydrogel is flowable at about 4° C. and solidifies after topical application to skin. In some embodiments, the ROS-scavenging polymer-containing hydrogel comes a high viscosity mass, e.g. solid or semi-solid mass, at about 18±2° C. or higher, about 18° C. or higher, about 20° C. or higher, about 22° C. or higher, about 25° C. or higher, about 30° C. or higher, about 37° C. or higher.

In some embodiments the boronate ester-based copolymer is a terpolymer comprising one or more acrylamide monomer(s), one or more methacrylate monomer(s), and one or more boronate ester acrylate monomer(s). In some embodiments, acrylamide (AM) monomer(s) refers to (N-alkyl) acrylamide (NAAM) and homologs or analogs thereof, e.g. N-isopropylacrylamide, N-isobutylacrylamide, N-n-propylacrylamide, N-isopropylmethylacrylamide and other modified N-isopropylacrylamides. In some embodiments, methacrylate (MAc) monomer(s) refers to (hydroxyalkyl) methacrylate (HAMc) and homologs or analogs thereof, e.g. 4-hydroxypropyl methacrylate, 2-hydroxyethyl acrylate, hydroxypolyethoxy allyl ether, hydroxypropyl methacrylate, 3-phenoxy 2 hydroxy propyl methacrylate, 2-hydroxyethyl acrylate, glycerol monomethacrylate and N-(2-hydroxypropyl) methacrylamide and other esters formed by (methyl) acrylic acid and diols. In some embodiments, boronate ester acrylate ester (BEAc) monomer(s) refers to 4-(hydroxyalkyl)-phenylboronic acid, pinacol ester) acrylate (HPPE-Ac) and homologs or analogs thereof, e.g. ethyl 4-(4,4,5,5-Tetramethyl-1,3,2-dioxaborolan-2-yl)benzoate, 2-Ethoxycarbonylphenylboronic acid pinacol ester, 3-Ethoxycarbonylphenylboronic acid pinacol ester, 4-(Acetoxymethyl)benzene boronic acid pinacol ester, 3-(4,4,5,5-tetramethyl-1,3,2-dioxaborolan-2-yl)propyl Benzoate. Suitable alkyl chain length is about C3-C8.

The AM monomer and homologs and analogs thereof are defined by the following chemical structure:

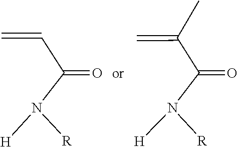

wherein R is propyl, isopropyl, butyl, isobutyl or other alkyl group of 5 or less carbon atoms in length.

The MAc monomer and homologs and analogs thereof are defined by the following chemical structure:

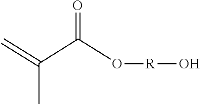

wherein R is (CH₂)ₙ; and n=2, 3, or 4.

The BEAc monomer and homologs and analogs thereof are defined by the following chemical structure:

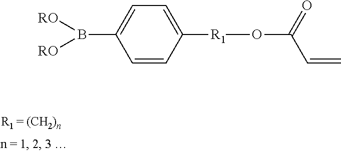

In some embodiments, the (boronate ester) acrylate ester is a (phenylboronate ester) acrylate ester homolog or analog. It should be understood that other boronate esters, aside from the pinacol ester, would be suitable for use. The preferred boronate esters are selected from the group consisting of pinacol ester, boronic acid cyclic monoester, 1,3-propanediol ester, 2,4-pentanediol ester, or neopentylglycol ester.

The boronate ester-based copolymer is also referred to herein as poly((AM)ₘ-co-(MAc)ₙ-co-(BEAc)ₓ), wherein: m is in the range of about 75 to about 85, n is in the range of about 5 to about 20, and x is in the range of about 5 to about 20. The boronate ester-based copolymer can also be referred to herein as poly((NAAM)ₘ-co-(HAMAc)ₙ-co-(HPPE-Ac)ₓ), wherein: m, n, and x are as defined herein.

In some embodiments, the boronate ester-based copolymer excludes an alkyne group-containing monomer, and thereby excludes an alkyne group.

In some embodiments, alkyl is independently selected upon each occurrence from the group consisting of (C₁-C₂)-alkyl or (C₃-C₈)-alkyl, which alkyl may be linear, branched, or cyclic. For example, the alkyl groups can be C₁-C₆ linear and branched alkyl. Other groups can be added into the system as the main functional group.

In some embodiments, the ROS-scavenging polymer (AAHPPE) comprises a copolymer of NIPAA, HEMA, and AHPPE, wherein NIPAAM is N-isopropylacrylamide, HEMA is hydroxyethyl methacrylate, AHPPE is (4-(hydroxymethyl)-phenylboronic acid, pinacol ester) acrylate (2-Propenoic acid or [4-(4,4,5,5-tetramethyl-1,3,2-dioxaborolan-2-yl) phenyl]methyl ester), and AAHPPE is poly (N-isopropylacrylamide-co-hydroxyethyl methacrylate-co-2-Propenoic acid). The AAHPPE gel is also referred to herein as poly((NIPAA)ₘ-co-(HEMA)ₙ-co-(AHPPE)ₓ), wherein: m, n, and x are as defined herein.

The invention also provides a method of preparing a boronate ester acrylate monomer (BEAc) comprising: reacting AM (or homolog or analog thereof) with acryloyl chloride (or homolog or analog thereof) in the presence of an amine catalyst to form said BEAc (or homolog or analog thereof). Said reacting can be conducted with cooling and/or at about room temp. The amine catalyst can be tertiary amine, such as triethanolamine (TEA), dimethylethylamine (DEMA), N-methyl morpholine (NMM), ditertiary amines triethylene diamine (DABCO), trimethylpropane diamine (TMP(an)DA) and trimethylpropene diamine (TMP(en)DA), or aromatic amine, such as pyridine, 4-Dimethylaminopyridine (DMAP), morpholine, n-C₄H₉NH₂ and DBU (1,8-Diazabicyclo[5.4.0]undec-7-ene). The invention also provides the intermediate BEAc (or analog thereof).

The following is an exemplary reaction scheme for preparing said monomer.

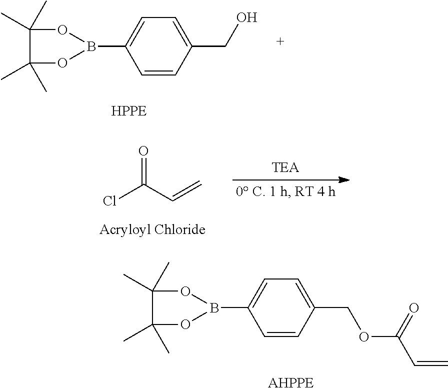

The invention also provides a method of preparing a boronate ester copolymer, the method comprising a) providing one or more AM monomer(s), one or more MAc monomer(s), and one or more BEAc monomer(s); and b) reacting said monomers in the presence of peroxide catalyst to form said gel. In some embodiments, the molar ratio of AM:MAc:BEAc is about 75-85 (AM): about 5-20 (MAc): about 5-20 (BEAc), or about 75-82 (AM): about 10-18 (MAc): about 6-18 (BEAc), or about 75-80 (AM): about 12-15 (MAc): about 6-16 (BEAc). In some embodiments, the molar ratio of AM:MAc:BEAc is about 75: about 15: about 10, or about 77: about 15: about 8, or about 79: about 15: about 6.

Said reacting can be conducted above room temp or with heating, e.g. about 50°-80° C. or about 65° C. Said peroxide catalyst (initiator) can be benzoyl peroxide (BPO), or Azobisisobutyronitrile (AIBN). The amount of initiator (catalyst) can influence the molecular weight of the final polymer. The content of initiator in the reaction milieu is usually less than about 5%, less than about 4%, less than about 3%, less than about 2%, less than about 1%, less than about 0.5%, or about 0.15% to about 0.35%, wherein the percentage is with respect to the total molar content of the monomers. The minimum content of initiator is at least about 0.01%, at least about 0.05%, at least about 0.1%, or at least about 0.15%, wherein the percentage is with respect to the total molar content of the monomers.

The following is an exemplary reaction scheme for preparing said boronate ester-based copolymer.

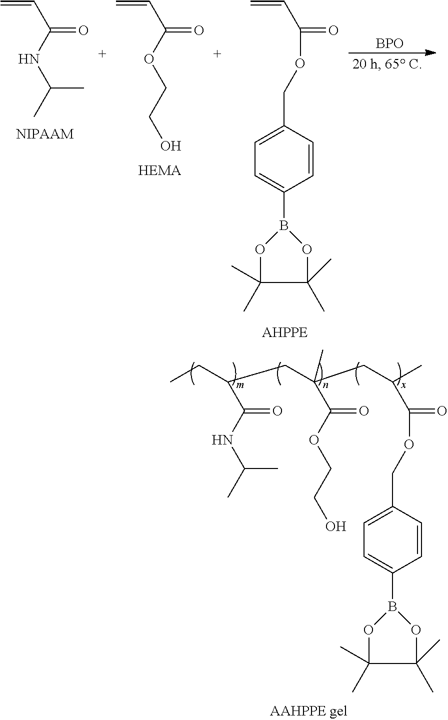

Different grades of the boronate ester-based copolymer differing in the molar content of the monomers were prepared. Aqueous hydrogels were prepared from those copolymers, which were then characterized according to injectability, water content, gelation temperature (LCST), and responsiveness to oxidative stress.

Some of the grades are described according to the following molar content (m, n, x) of the respective monomers.

| | m (NIPAAm) | n (HEMA) | x (AHPPE) |
|---|---|---|---|
| AHPPE-6 | 79 | 15 | 6 |
| AHPPE-8 | 77 | 15 | 8 |
| AHPPE-10 | 75 | 15 | 10 |

Physicochemical characterization of the aqueous hydrogels made from the boronate ester-based copolymer grades provided the following.

| | | Water | LCST |
|---|---|---|---|
| Hydrogel | Injectability | content (%) | (° C.) |
| AHPPE-6 | + | 82.4 ± 4.7 | 27.6 ± 1.9 |
| AHPPE-8 | + | 75.6 ± 5.3 | 22.6 ± 3.8 |
| AHPPE-10 | + | 73.2 ± 5.1 | 18.5 ± 3.1 |

Accordingly, decreasing the molar content of the NIPAAm monomer and increasing the molar content the AHPPE monomer caused the hydrogel to have a lower water content and a lower gelation temperature.

Responsiveness to oxidative stress (with H₂O₂) was evaluated according to Example 7. The degradation of AHPPE gels (in DPBS) at 37° C. for 4 weeks without and with 50 mM hydrogen peroxide (H₂O₂) showed that the gels comprised of different AHPPE compositions exhibited high responsiveness to oxidative stress. The data (FIGS. 12A-12C) indicate that decreasing the molar content of the NIPAAm monomer and increasing the molar content the AHPPE monomer caused the gel to exhibit greater responsiveness to oxidative stress.

The content of boronate ester-based copolymer in the cosmeceutical composition can vary widely according to the desired clinical (cosmetic) effect. In some embodiments, the boronate ester-based copolymer is present in the cosmeceutical composition at a concentration of up to about 10% wt or at a range of about 4% to about 10% wt. The remaining content of the cosmeceutical comprises liquid vehicle (in an amount sufficient to render a syringeable (low viscosity) hydrogel) and optionally one or more cosmeceutical excipients. The content of liquid vehicle in the hydrogel can be at least about 30% wt, at about least 40% wt, at about least 50% wt, at about least 60% wt, at about least 70% wt, at about least 80% wt, or at about least 90% wt.

A ready-to-use cosmetic composition comprising the boronate ester-based copolymer and a cosmetic vehicle (cosmetic excipient) can be prepared by dissolving the copolymer in DPBS (other substituents like DI water, saline and cell culture medium) to form a hydrogel solution. Even in the presence of a liquid cosmetic vehicle, the gel solution exhibits thermosensitivity. Usually, the gel solution concentration in DPBS (or water, saline and medium) ranges from about 4 wt % to about 10 wt % (0.04 g˜0.1 g copolymer in 1 mL of vehicle).

A composition of the invention may comprise a ROS-scavenging polymer in admixture with one or more excipients.

A composition of the invention may comprise a ROS-scavenging polymer in admixture with one or more antioxidants and one or more excipients.

A composition of the invention may comprise MG53 in admixture with one or more excipients.

A composition of the invention may comprise a ROS-scavenging polymer in admixture with MG53 and one or more excipients.

A composition of the invention may comprise a ROS-scavenging polymer in admixture with one or more antioxidants, MG53, and one or more excipients.

A composition of the invention may comprise a ROS-scavenging polymer in admixture with one or more antioxidants, MG53, one or more zinc salts, and one or more excipients.

A composition of the invention may comprise one or more antioxidants, MG53, one or more zinc salts, and one or more excipients.

A composition of the invention may comprise MG53, one or more zinc salts, and one or more excipients.

A composition of the invention may be present as a gel, cream, or ointment. The composition may be adapted for topical administration, dermal administration, transdermal administration, subcutaneous administration.

An MG53-containing composition of the invention exhibits improved stability and/or improved efficacy as compared to a saline-based aqueous composition comprising the same amount of MG53.

A combination composition comprising MG53, ROS-scavenging copolymer of the invention, and one or more excipients provides a synergistic clinical benefit. In some embodiments, the combination composition provides improved wound healing as evidenced by faster wound size reduction as compared to that provided by administration of just MG53 or of just the ROS-scavenging copolymer. In some embodiments, the combination composition provides reduced collagen formation (aggregation) at the site of a healed wound as compared to that provided by administration of just MG53 or of just the ROS-scavenging copolymer. In some embodiments, the combination composition provides improved increased follicle density at the site of a healed wound as compared that provided by to administration of just MG53 or of just the ROS-scavenging copolymer.

One or more cosmeceutical excipients and/or one or more pharmaceutical excipients and/or one or more GRAS excipients can be added to any composition of the invention.

An aspect of the invention also provides a method of improving the appearance of skin by administering to the skin a cosmetically effective amount of composition of the invention. In some embodiments, the improvement is selected from the group consisting of reducing wrinkles, reducing irritation, reducing inflammation, reducing redness, reducing scarring, reducing collagen formation, reducing differentiation of stem cells into fibroblasts, reducing the period of time for amelioration of dermal of epidermal condition(s).

An aspect of the invention also provides a method of preventing ROS-related oxidative damage to skin by prophylactically administering to the skin a cosmetically effective amount of composition of the invention prior to exposure of said skin to the ROS.

An aspect of the invention also provides a method of sequestering, neutralizing or eliminating ROS in skin comprising administering to the skin a cosmetically effective amount of composition of the invention after exposure of said skin to the ROS.

An aspect of the invention also provides a method of improving hair growth by administering to the skin of a subject in need thereof a cosmetically effective amount of composition of the invention. Said improved hair growth may include, by way of example and without limitation, increased hair strand thickness, improved hair follicle health, increased number of hair follicles per unit area of skin, increased rate of hair growth, improve the health of hair. The composition of the invention can be used to reduce baldness.

An aspect of the invention also provides a method of removing or improving the appearance of skin blemishes by administering to the skin of a subject in need thereof a cosmetically effective amount of composition of the invention. In some embodiments, the blemish is selected from the group consisting of wrinkle(s), blotch(es), reddened patch(es), and eczema.

An MG53-containing composition of the invention provides improved healing of diabetic ulcers and diabetes-related skin conditions as compared to a saline-based aqueous composition containing the same amount of MG53.

The invention also provides a cosmeceutical gel comprising at least one cosmetically acceptable ROS-scavenging polymer and at least one liquid vehicle, wherein said gel exhibits low viscosity at about 4° C. and solidifies or semi-solidifies (thicken substantially into a high viscosity mass) after contact with skin, or after contact with a surface of at least about 18° C., at least about 20° C., or at least about 25° C. The cosmeceutical gel may comprise any one or more of the compositions of the invention.

The invention also provides an extended release cosmeceutical composition comprising at least one ROS-scavenging polymer and at least one active ingredient, wherein said at least one active ingredient is released from said composition over a period of at least about 3 h, at least about 6 h, at least about 9 h, at least about 12 h, at least about 15 h, at least about 18 h, or at least about 21 h. In some embodiments, said at least one active ingredient is released from said composition over a period of at least about 1 day, at least about 2 days, at least about 3 days, at least about 4 days, at least about 5 days, at least about 6 days, at least about 1 week, at least about 8 days, at least about 9 days, at least about 10 days, at least about 11 days, at least about 12 days, at least about 13 days, at least about 2 weeks, at least about 3 weeks, or at least about 4 weeks.

Another aspect of the invention provides a method of healing injured tissue, said method comprising administering to said injured tissue an effective amount of ROS-scavenging polymer. In some embodiments, the ROS-scavenging polymer is present in a hydrogel further comprising a liquid vehicle.

Another aspect of the invention provides a cosmeceutical gel comprising at least one cosmetically acceptable antioxidant polymer, an effective amount of MG53, and at least one liquid vehicle, wherein said gel exhibits a low viscosity at less than about 15° C., and said gel solidifies or semi-solidifies (thicken substantially into a high viscosity mass) after contact with skin, or after contact with a surface of at least about 18° C., at least about 20° C., or at least about 25° C.

Another aspect of the invention provides a cosmeceutical cream comprising a cream base and an effective amount of MG53. Said cream can further comprise one or more active ingredients. Said cream base can comprise one or more cosmetically acceptable (and/or pharmaceutically acceptable) excipients. Exemplary active ingredients and excipients include, by way of example and without limitation, Acai oil, Alpha-lipoic acid, Green tea extract, Retinol, Coenzyme Q10 (CoQ10), Caffeine, Alpha-hydroxy Acids (AHAs), Hyaluronic Acid, Glycerin, Sorbitol, Mono and Diglycerides, White Mineral Oil (Petroleum Hydrocarbon Oil), Glyceryl Stearate, Propylene Glycol, Vitamin E, Citric Acid, Propyl Gallate, other known compounds, and combinations thereof.

Another aspect of the invention provides a cosmeceutical ointment comprising an ointment base and an effective amount of MG53.

In some embodiments, the skin is not suffering from a disease. In some embodiments, the skin has not been physically injured, such as by impact force, burning, irradiation, or cutting. In some embodiments, the tissue is otherwise healthy except for exhibiting cosmetically undesired features.

It should be understood that some embodiments of the invention that include MG53 rely primarily on administration of exogenous MG53, optionally along with one or more antioxidant(s), boronate ester-based copolymer gel, or combination thereof.

The composition may be administered one or more times over a treatment period of at least one week. The composition may be administered acutely or chronically. In some embodiments, the chronic administration is at least one weekly, at least once daily, two or more times daily, two or more times per week, or as needed at a dose of about 0.01 mg of MG53/kg of bodyweight to about 10 mg of MG53/kg of bodyweight. The composition can be applied plural times within a day and for plural days.

Exemplary subjects that can be treated with compositions of the invention include humans and animals.

In some embodiments, the method of the invention further comprises adjunct administration of with at least one antioxidant, whereby said at least one antioxidant is administered prior to, along with, or after administration of MG53. Accordingly, the method of the invention can further comprise the step of administering at least one antioxidant to a subject. The molar ratio of MG53 to antioxidant can be in the range of 0.01:1 to 10:1.

When administered prophylactically, MG53 can prevent the appearance of cosmetically undesirable features of the skin.

A composition of the invention can be administered one, two, three or more times per day. It can be administered daily, weekly, monthly, bimonthly, quarterly, semiannually, annually or even longer as needed. It can be administered every other day, five times per week, four times per week, three times per week, two times per week, once daily, twice daily, one to four times daily, continuously, or as frequently or infrequently as needed. The unit dose of each administration is independently selected upon each occurrence from the doses described in this specification or as determined to be therapeutically effective. All combinations of the dosing regimens described are contemplated to be within the scope of the invention.

In some embodiments, a composition of the invention comprises about 1.5 mg or more of MG53 per ounce of composition. A composition may comprise about 1.5 to about 50 mg/oz, or about 1.5 to about 40 mg/oz, or about 1.5 to about 30 mg/oz, or about 1.5 to about 20 mg/oz, or about 1.5 to about 5 mg/oz, or about 1.5 to about 3 mg/oz of composition.

An MG53-containing composition can be administered topically or topically in combination with systemically.

Another aspect of the invention provides a co-therapeutic or adjunctive method of improving tissue performance, the method comprising administering to a subject in need thereof (meaning a subject with one or more cosmetically undesirable features) an effective amount of MG53 and an effective amount of one or more other active ingredients, which are suitable for improving the appearance of skin. Exemplary other active ingredients include Acai oil, Alpha-lipoic acid, Green tea extract, Retinol, Coenzyme Q10 (CoQ10), Caffeine, Alpha-hydroxy Acids (AHAs), Hyaluronic Acid, other known compounds, and/or combinations thereof. MG53 and said one or more other active ingredients can be administered simultaneous, sequentially or in an overlapping manner.

In some embodiments, the composition further comprises one or more zinc salts present in an amount sufficient to stabilize MG53 present in the composition.

In a composition of the invention, the molar ratio of Zn ions present to MG53 molecules present is at least 2:1, when considering the two zinc ion binding sites present on each MG53 molecule. In some embodiments, the composition comprises a molar ratio of >2:1 for the moles of Zn to moles of MG53.

In some embodiments, a subject is chronically administered MG53, at least one antioxidant, and at least one zinc salt. The invention also provides a composition comprising MG53, at least one antioxidant, and at least one zinc salt.

The molar ratio of MG53 to antioxidant can be in the range of 0.01:1 to 10:1.

Embodiments of the invention exclude compositions comprising single unaltered natural product; however, said compositions may comprise mixtures of said unaltered natural product(s) along with other components thereby resulting in manmade compositions not present in nature. Embodiments of the invention exclude processes that employ solely unaltered natural processes; however, said processes may comprise a combination of said unaltered natural processes along with one or more other non-natural steps, thereby resulting in processes not present in nature. Embodiments of the invention may also include new uses (new methods of treatment) for natural products, new compositions comprising said natural products, and new methods employing said natural products.

The invention includes all combinations of the aspects, embodiments and sub-embodiments disclosed herein. Other features, advantages and embodiments of the invention will become apparent to those skilled in the art by the following description, accompanying examples and appended claims.

### BRIEF DESCRIPTION OF THE FIGURES

The following drawings are part of the present specification and are included to further demonstrate certain aspects of the invention. The invention may be better understood by reference to one or more of these drawings in combination with the detailed description of the specific embodiments presented herein.

FIG. 1 depicts a chart of the number of hair follicles per unit area of skin for wild-type and MG53 knockout (mg53−/−) mice. Quantitation of hair follicles was conducted according to Example 15.

FIG. 2 depicts photographs of the mice of FIG. 1 twelve days after being shaved and then being administered saline or saline containing MG53 (dose of 1 mg/kg) Example 16.

FIG. 3 depicts photographs of rats thirty days after being shaved and then administered bovine serum albumin (BSA, dose of 1 mg/kg) in saline or MG53 (dose of 1 mg/kg) in saline (Example 17).

FIG. 4 depicts a chart of the number of hair follicles per unit area of skin for rats four days after being administered saline or saline containing MG53 (dose of 1 mg of MG53/kg; Example 17).

FIG. 5 depicts a chart of the number of apoptotic human hair follicle stem cells (HFSC's) twenty-four hours after treatment with hydrogen peroxide (100 micromolar), MG53 (1 microg/ml), or MG53 and hydrogen peroxide as compared to control (Example 18).

FIG. 6 depicts a chart of the number of hair follicles per unit area for diabetic mice sixteen days after treatment with cream base or cream containing MG53 (10 mg MG53/oz of cream base) (Example 19).

FIGS. 7A-7C depict chronological charts of degradation of various grades the copolymer after exposure to H₂O₂(50 mM).

FIG. 8 depicts a chart demonstrating the synergistic efficacy of the combined MG53 and boronate ester-based copolymer based upon wound size reduction over time.

FIG. 9 depicts a chart demonstrating the synergistic efficacy of the combined MG53 and boronate ester-based copolymer based upon collagen formation after healing of a wound.

FIG. 10 depicts a chart demonstrating the synergistic efficacy of the combined MG53 and boronate ester-based copolymer based upon follicle density after healing of a wound.

FIG. 11A depicts a chart of the relative change in melanin deposition after topical treatment of a subject's facial skin with MG53 containing mask according to Example 26.

FIG. 11B depicts a chart of the relative change in wrinkles after topical treatment of a subject's facial skin with MG53 containing mask according to Example 26.

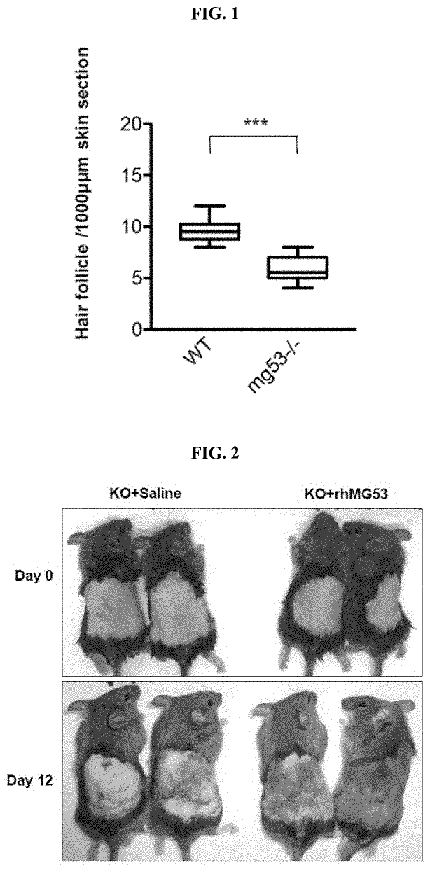

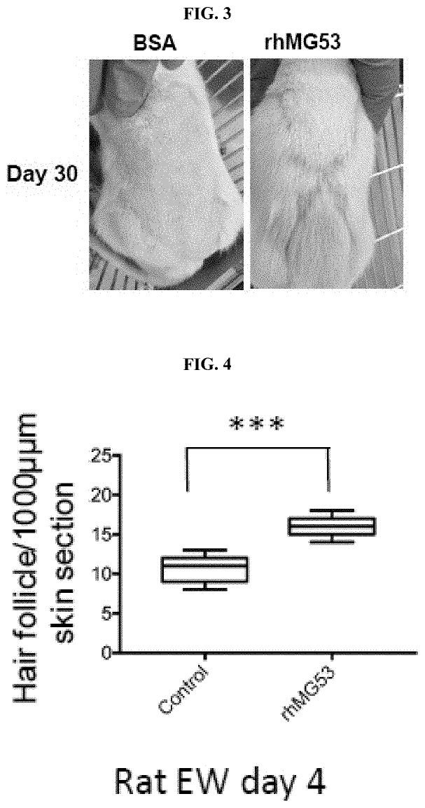

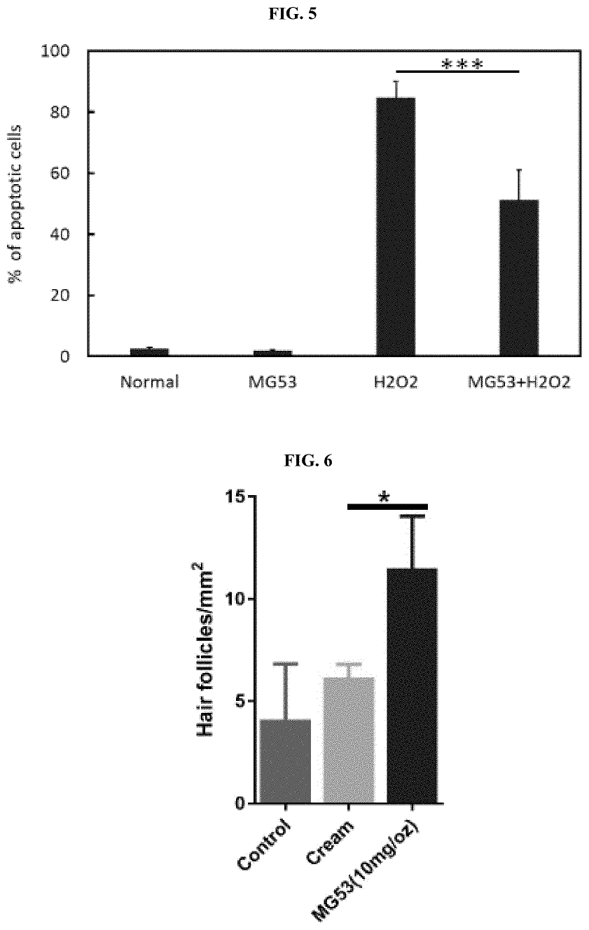

### DETAILED DESCRIPTION OF THE INVENTION

As used herein, the term ROS-scavenging polymer also refers to a reactive oxygen species-scavenging polymer, whereby such polymers can be more generally referred to as examples of an antioxidant polymer. ROS (reactive oxygen species) comprise both free radical and non-free radical oxygen intermediates such as hydrogen peroxide (H₂O₂), superoxide (O₂^{⋅-}), singlet oxygen (¹O₂), and the hydroxyl radical. Accordingly, the ROS-scavenging polymer of the invention is able to scavenge such oxygen intermediates so that they do not damage tissue.

MG53 protein (also referred to as mitsugumin 53 or TRIM72) is known in the art. Unless specified otherwise, all embodiments of the invention comprising or employing “MG53” include all known forms of MG53. It also refers to recombinant human MG53 (rhMG53). As used herein and unless otherwise specified, the term MG53 (or MG53 protein) refers to the MG53 protein present as the native form, optimized form thereof, mutant thereof, derivative thereof or a combination of any two or more of said forms. Native MG53 contains 477 amino acids that are well conserved in different animal species. Methods of preparing and/or isolating MG53 are known: U.S. Pat. No. 7,981,866, WO2008/054561, WO2009/073808, US2011/0202033, US2011/0287004, US2011/0287015, US2013/0123340, WO2011/142744, WO2012/061793, U.S. Pat. Nos. 8,420,338, 9,139,630, 9,458,465, 9,494,602, US2014/0024594, WO2012/134478, WO2012/135868, US2015/0110778, WO2013/036610, US2012/0213737, WO2016/109638, the entire disclosures of which, including sequence information therein, are hereby incorporated by reference.

The sequence listing information for native MG53, and variants or various forms thereof, is disclosed in U.S. Pat. Nos. 7,981,866 and 9,139,630, the entire disclosures of which, including sequence information therein, are hereby incorporated by reference. The sequence listing information for a cDNA that encodes optimized native human MG53, or a fragment thereof, is disclosed in U.S. Pat. No. 9,139,630, the entire disclosure of which, including sequence information therein, is hereby incorporated by reference.

As used herein in reference to MG53, the term “mutant” means a recombinant form of MG53 having an amino acid change (replacement) of one, two, three or more amino acids in the amino acid sequence of native MG53. Mutant forms of MG53 and methods of preparing the same are known: US2015/0361146, EP3118317, WO2015/131728, U.S. Pat. No. 9,139,630, the entire disclosures of which, including sequence information therein, are hereby incorporated by reference.

As used herein the term “endogenous MG53”, refers to MG53 present in a subject prior to treatment with a composition, cosmeceutical, or method according to the invention. As used herein, exogenous MG53 is nonendogenous MG53.

The present inventors have unexpectedly discovered that an antioxidant polymer applied to the skin can be used to improve the appearance and health of the skin. A boronate ester-based antioxidant polymer can react with ROS in the skin, and thereby prevent and/or reduce the extent of oxidative damage to the skin that is caused by said ROS. Moreover, addition of a cosmetically effective amount (concentration) of MG53 to said antioxidant polymer further improves the appearance of skin and the growth of hair.

The ROS-scavenging polymer of the invention is a boronate-ester based copolymer gel. It is prepared by reacting acrylamide (or homolog or analog thereof), methacrylate ester (or homolog or analog thereof), and (boronate ester) acrylate ester (or homolog or analog thereof). The molar ratio of the monomers in the reaction milieu approximates the molar ratio of the monomers in the resulting boronate-ester based copolymer: poly((AM)ₘ-co-(MAc)ₙ-co-(BEAc)ₓ), wherein: m, n and x are as defined herein. The identity of each monomer is independently selected upon each occurrence. Thus, the copolymer can comprise one or more acrylamide monomers, one or more methacrylate ester monomers, and one or more (boronate ester) acrylate ester monomers.

An exemplary boronate ester monomer was prepared according to Example 1. HPPE was dissolved in water immiscible organic solvent and the amine, in slight molar excess, was added thereto. Acryloyl halide, at about the same molar amount as the amine, was added dropwise over a period of time to the prior mixture. After completion of the reaction, the reaction milieu was washed with water and then brine. The organic layer was then dried over desiccant, separated therefrom, and the solvent removed under vacuum. The boronate ester monomer was obtained in high yield.

An exemplary antioxidant copolymer of the invention was prepared according to Example 2. The boronate ester monomer prepared according to Example 1 was used as the starting material for preparation of the copolymer. Generally, the monomers were mixed in an organic solvent and repeatedly purged with nitrogen. Initiator was then added to the mixture and the entire reaction milieu heated at 65° C. for 20 hours with continuous stirring. The copolymer was isolated by repeated precipitation-dissolution cycles using a mixture of solvents and recovery of solid by filtration prior to each dissolution and after each precipitation. The copolymer with then dried under vacuum with heat. The copolymer was obtained in high yield.

The liquid gel, comprising the antioxidant (ROS-scavenging) copolymer and an aqueous vehicle, was obtained by dissolving the copolymer in the aqueous vehicle while stirring overnight at about 4° C. (Example 3). The content of copolymer in the gel was about 6 wt % and the content of aqueous vehicle was about 94%.

Before solidification, a hydrogel solution of the copolymer (about 6 wt % in aqueous vehicle) has a viscosity of less than about 0.1 Pa·sec. Solidification of the hydrogel solution was performed according to Example 3. A portion of hydrogel solution was placed in a tube which was then placed in a heated water bath at 37° C. The gel solidified within seconds. After solidification, the solidified mass viscosity was high.

The novel antioxidant polymer reduces oxidative damage caused to the skin (tissue) by ROS. A composition comprising antioxidant polymer and one or more antioxidants provides even greater efficacy. A composition comprising antioxidant polymer, MG53, and one or more antioxidants provides still greater efficacy.

As part of the synthetic pathway toward the target antioxidant copolymer, we have also invented the novel intermediate (or monomer) (4-(hydroxymethyl)-phenylboronic acid, pinacol ester) acrylate. It should be understood that structural homologs and analogs thereof are contemplated within the scope of the invention. Accordingly, the group of monomers defined herein are contemplated.

The molar ratio of the three monomers in the copolymer may impact performance. The hydrophilicity of the gel increases as the molar ratio increases for MAc or molar ratio decreases for AM/BEAc. As the hydrophilicity changes, the mechanical properties including flexibility, elastic modulus, complex modulus, tensile strength and Young's modulus will change accordingly. For example, a more hydrophilic structure in the copolymer will cause a decrease in tensile strength and modulus as the gels become softer.

At about 4° C., the antioxidant polymer exhibits low enough viscosity to be syringeable by hand without mechanical assistance through an 18-30-gauge needle. When the antioxidant polymer is warmed to at least 35° C., it forms a much higher viscosity solid or semi-solid gel mass. For example, when the cooled gel is applied as a layer topically to human skin, which is at about 35-38° C., the gel forms a solid or semi-solid layer.

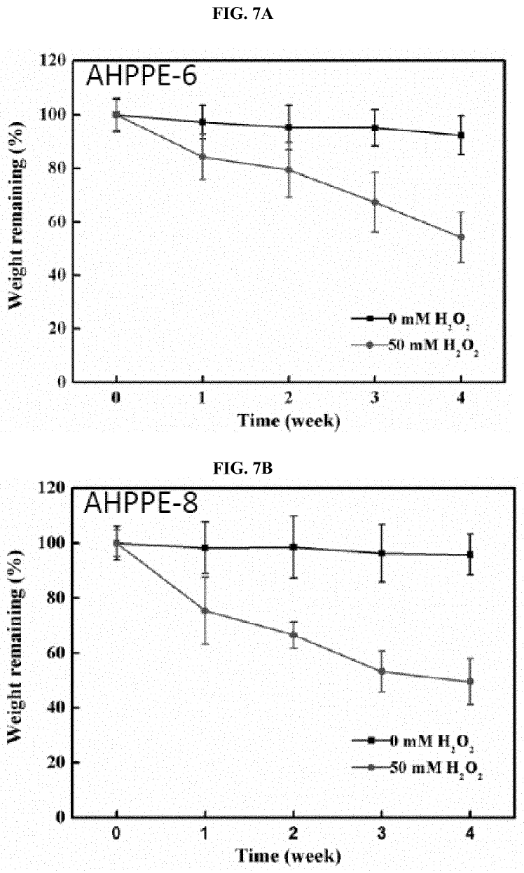

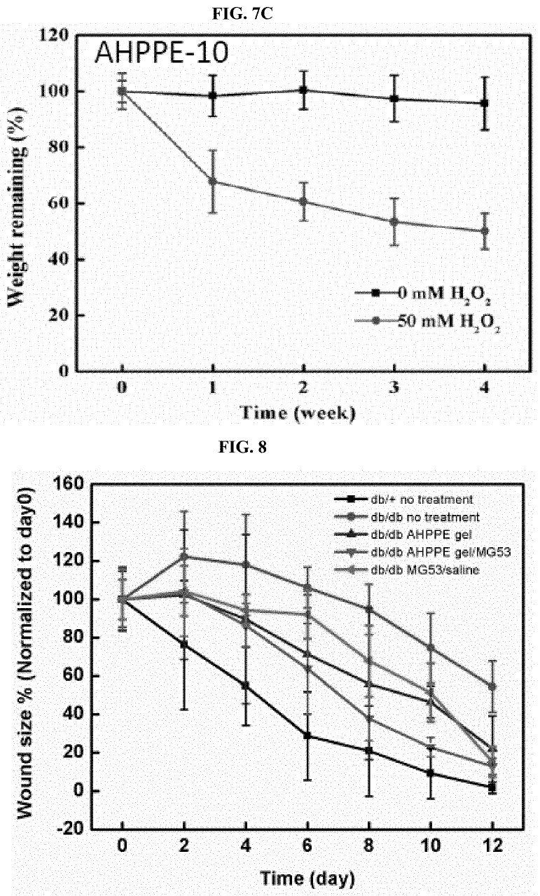

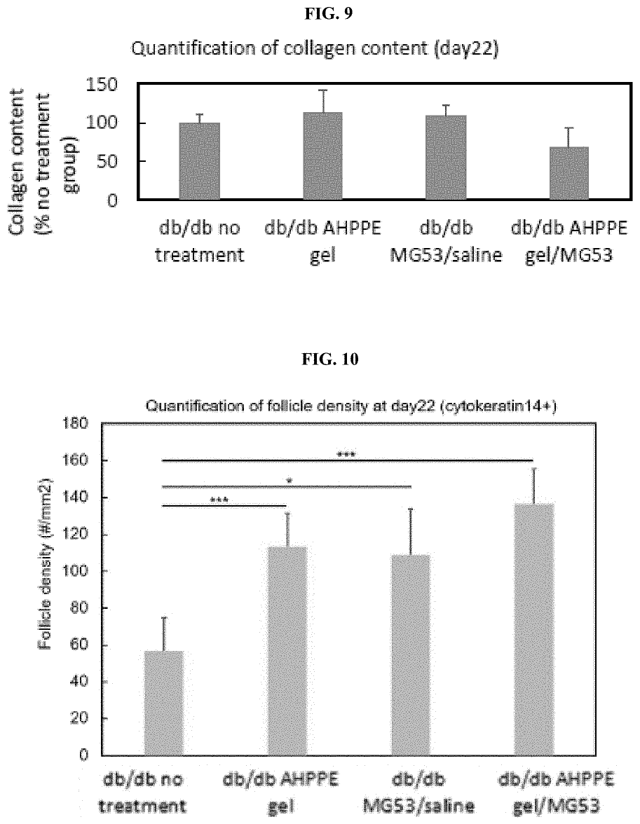

The compositions of the invention are suitable for use as wound dressings. The ROS-scavenging copolymer (optionally in admixture with MG53) can be administered to a wound in dry form, whereby it will absorb body fluid and become a hydrogel that then solidifies at the site of the wound. The ROS-scavenging copolymer (optionally in admixture with MG53) can be administered to a wound in hydrogel form, whereby it then solidifies at the site of the wound. The copolymer provides reduced collage formation (FIG. 8), increased follicle density (FIG. 9), and faster wound size reduction (FIG. 7) as compared to an untreated wound. The copolymer in combination with MG53 provides even further reduced collage formation, further increased follicle density, and even faster wound size reduction as compared to an untreated wound.

The ROS-scavenging hydrogel accelerates wound healing due to its function in downregulating the overproduction of ROS on chronic wounds. An appropriate amount of hydrogel at a concentration of 6 wt % administered to a 5 mm-wound on mice ranges from about 150 μL to about 250 μL.

Suitable concentrations of MG53 in a dosage form include at least 1 ng of MG53/ml, at least 5 ng of MG53/ml, at least 10 ng of MG53/ml, at least 25 ng of MG53/ml, at least 50 ng of MG53/ml, at least 75 ng of MG53/ml, at least 100 ng of MG53/ml, at least 250 ng of MG53/ml, at least 500 ng of MG53/ml, at least 750 ng of MG53/ml, at least 1 μg of MG53/ml, at least 5 μg of MG53/ml, at least 10 μg of MG53/ml, at least 15 μg of MG53/ml, at least 20 μg of MG53/ml, at least 25 μg of MG53/ml, at least 30 μg of MG53/ml, at least 50 μg of MG53/ml, or at least 100 μg of MG53/ml. Higher concentrations are also acceptable, particularly in view the efficacy dose-response trend observed for MG53. These doses can be administered on a frequency as described herein or as determined to be most effective.

Suitable doses of MG53 that can be administered to a subject in one or more dosage forms include at least 1 ng of MG53, at least 5 ng of MG53, at least 10 ng of MG53, at least 25 ng of MG53, at least 50 ng of MG53, at least 75 ng of MG53, at least 100 ng of MG53, at least 250 ng of MG53, at least 500 ng of MG53, at least 750 ng of MG53, at least 1 μg of MG53, at least 5 μg of MG53, at least 10 μg of MG53, at least 15 μg of MG53, at least 20 μg of MG53, at least 25 μg of MG53, at least 30 μg of MG53, at least 50 μg of MG53, or at least 100 μg of MG53. Such doses can be on a total body weight basis or a per kg of body weight basis.

The dose of exogenous MG53 can be as low as about 1 up to about 1000 microg per kg of body weight.

The efficacy of the cosmeceutical composition was established in vivo. The face of a person having undergone laser ablation was subsequently treated with MG53-containing cream (according to Example 13) on only one side of the face according to Example 20 as compared to the standard of care which is a currently marketed conventional moisturizer. The creams were applied daily for seven days. After four days of daily application, the side of the subject's face treated with MG53 was less swollen and exhibited less redness as compared to the conventional moisturizer cream.

An elderly person having red blistering of unknown etiology was treated with MG53-containing cream (according to Example 13). After three days of daily application (Example 21), the subject's face exhibited reduced blistering as compared to the conventional moisturizer cream. MG53 cream application has significantly reduced time to healing by at least 1 week. This rate of healing was more rapid with daily application AND coverage with bandage. Therefore, keeping the wound moist with MG53 and covered allowed faster epithelialization of the wounds. Furthermore, areas where it is applied regularly (i.e. wrist) are less prone to tearing and blistering.

An elderly person with wrinkled skin along the cheeks and neck was treated with MG53-containing cream (according to Example 13). The creams were applied for 28 days. After three weeks of daily application (Example 22), the subject's cheeks and neck exhibited substantially reduced wrinkles (both reduced depth and reduced appearance of wrinkles) as compared to the conventional moisturizer cream.

An elderly person with wrinkled skin along the lips and eyes was treated with MG53-containing cream (according to Example 13). The creams were applied for 28 days. After three weeks of daily application (Example 22), the subject's lips and eyes exhibited substantially reduced wrinkles (both reduced depth and reduced appearance of wrinkles) as compared to the conventional moisturizer cream.

An elderly person with a long-term chronic nonhealing wound on the skin was treated with MG53-containing cream (according to Example 13). After three weeks of daily application (Example 23), the skin was completed healed and exhibited no scarring. Conventional moisturizer cream was ineffective.

A 60-year old male underwent a cryofreeze procedure to remove large moles from the left temple. The cryofrozen sites were treated 2-3 times daily with MG53-containing cream (according to Example 13). After 1 week of treatments, the skin was completed healed and exhibited no scarring.

The invention also provides an MG53-containing cream comprising water (about 65-75%% w/w), at least one preservative (about 0.1-1% w/w), at least one humectant (about 3-5% w/w), at least one exfoliant about 1-5% w/w), at least one penetration enhancer (about 5-15% w/w), at least one emollient (about 1-7% w/w), at least one protectant (skin conditioning agent; about 0.1-5% w/w), at least one antimicrobial (about 0.1-5% w/w), at least one zinc salt (about 0.1-1% w/w).

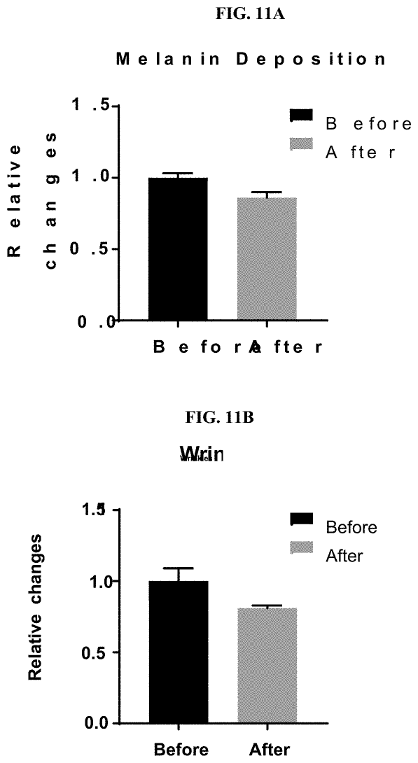

MG53 was also evaluated for use in reducing melanin formation and reducing wrinkles according to Example 26. Subjects were treated two to three times per week, for two to three weeks, with a facial mask saturated with normal saline solution comprising rhMG53 (20 microg/mL). In just two weeks, each subject exhibited a substantial reduction in melanin deposition (FIG. 11A) and wrinkles (FIG. 11B).

Accordingly, the invention provides a method of reducing skin wrinkles by topical administration of rhMG53. The invention also provides a method of reducing melanin deposition by topical administration of rhMG53.

The amount of therapeutic compound (MG53) incorporated in each dosage form will be at least one or more unit doses and can be selected according to known principles of pharmacy. An effective amount of therapeutic compound is specifically contemplated. By the term “effective amount”, it is understood that, with respect to, for example, pharmaceuticals, a pharmaceutically (therapeutically) effective amount is contemplated. A pharmaceutically effective amount is the amount or quantity of a drug or pharmaceutically active substance which is sufficient to elicit the required or desired therapeutic response, or in other words, the amount which is sufficient to elicit an appreciable biological response when administered to a patient.

The term “unit dosage form” is used herein to mean a dosage form containing a quantity of the drug, said quantity being such that one or more predetermined units may be provided as a single therapeutic administration.

The dosage form is independently selected at each occurrence from the group consisting of liquid solution, suspension, gel, cream, ointment, slab, gel, insert (implant), syringe, and hydrogel.

Compositions and dosage forms of the invention can further comprise one or more pharmaceutically acceptable and/or cosmetically acceptable excipients. Dosage forms can comprise one or more excipients independently selected at each occurrence from the group consisting of acidic agent, alkaline agent, buffer, tonicity modifier, osmotic agent, water soluble polymer, water-swellable polymer, thickening agent, complexing agent, chelating agent, penetration enhancer. Suitable excipients include U.S.F.D.A. inactive ingredients approved for use in parenteral or oral formulations (dosage forms), such as those listed in the U.S.F.D.A.'s “Inactive Ingredients Database (available on the following website: www.fda.gov/Drugs/InformationOnDrugs/ucm113978.htm; October 2018), the entire disclosure of which is hereby incorporated by reference.

As used herein, an acidic agent is a compound or combination of compounds that comprises an acidic moiety. Exemplary acidic agents include organic acid, inorganic acid, mineral acid and a combination thereof. Exemplary acids include hydrochloric acid, hydrobromic acid, sulfuric acid, sulfonic acid, sulfamic acid, phosphoric acid, or nitric acid or others known to those of ordinary skill; and the salts prepared from organic acids such as amino acids, acetic acid, propionic acid, succinic acid, glycolic acid, stearic acid, lactic acid, malic acid, tartaric acid, citric acid, ascorbic acid, pamoic acid, maleic acid, hydroxymaleic acid, phenylacetic acid, glutamic acid, benzoic acid, salicylic acid, sulfanilic acid, 2-acetoxybenzoic acid, fumaric acid, toluenesulfonic acid, methanesulfonic acid, ethane disulfonic acid, oxalic acid, isethionic acid, others acids known to those of ordinary skill in the art, or combinations thereof.

As used herein, an alkaline agent is a compound or combination of compounds that comprises an alkaline moiety. Exemplary alkaline agents include primary amine, secondary amine, tertiary amine, quaternary amine, hydroxide, alkoxide, and a combination thereof. Exemplary alkaline agents include ammonia solution, ammonium carbonate, diethanolamine, monoethanolamine, potassium hydroxide, sodium borate, sodium carbonate, sodium bicarbonate, sodium hydroxide, triethanolamine, diethanolamine, monobasic phosphate salt, dibasic phosphate salt, organic amine base, alkaline amino acids and trolamine, others known to those of ordinary skill in the art, or combinations thereof.

Exemplary excipients (inactive ingredients as defined by the U.S.F.D.A.) that can be included in dosage forms of the invention include, by way of example and without limitation, water, benzalkonium chloride, glycerin, sodium hydroxide, hydrochloric acid, boric acid, hydroxyalkylphosphonate, sodium alginate, sodium borate, edetate disodium, propylene glycol, polysorbate 80, citrate, sodium chloride, polyvinylalcohol, povidone, copovidone, carboxymethylcellulose sodium, Dextrose, Dibasic Sodium Phosphate, Monobasic Sodium Phosphate, Potassium Chloride, Sodium Bicarbonate, Sodium Citrate, Calcium Chloride, Magnesium Chloride, stabilized oxychloro complex, Calcium Chloride Dihydrate, Erythritol, Levocarnitine, Magnesium Chloride Hexahydrate, Sodium Borate Decahydrate, Sodium Citrate Dihydrate, Sodium Lactate, Sodium Phosphate (Mono- and Dibasic-), Polyethylene Glycol 400, Hydroxypropyl Guar, Polyquaternium-1, Zinc Chloride, white petrolatum, mineral oil, hyaluronic acid, artificial tear, or combinations thereof.

One or more antioxidants can be included in a composition of dosage form of the invention. Exemplary antioxidants include SS-31, NAC, glutathione, selenium, vitamin A, vitamin C, vitamin E, co-enzyme Q10, resveratrol, other GRAS antioxidant, or a combination of two or more thereof.

One or more zinc salts can be included in a composition or dosage form of the invention. Such zinc salt(s) may also be administered to a subject receiving exogenous MG53 or expressed MG53. Pharmaceutically acceptable zinc salts include Zinc gluconate, Zinc acetate, Zinc sulfate, Zinc picolinate, Zinc orotate, Zinc citrate, and other such salts comprising a zinc cation and organic or inorganic anion(s).

It should be understood that compounds used in the art of pharmaceutical formulations generally serve a variety of functions or purposes. Thus, if a compound named herein is mentioned only once or is used to define more than one term herein, its purpose or function should not be construed as being limited solely to that named purpose(s) or function(s).

As used herein, “pharmaceutically acceptable salts” refer to derivatives of the disclosed compounds wherein the compound is modified by making an acid or base salt thereof. Examples of pharmaceutically acceptable salts include, but are not limited to, mineral or organic acid salts of basic residues such as amines; alkali or organic salts of acidic residues such as carboxylic acids; and others known to those of ordinary skill. The pharmaceutically acceptable salts can be synthesized from the parent therapeutic compound which contains a basic or acidic moiety by conventional chemical methods. Lists of suitable salts are found in Remington's Pharmaceutical Sciences, 17th ed., Mack Publishing Company, Easton, Pa., 1985, p. 1418, the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

MG53 can be used in cotherapy or adjunctive therapy with one or more other active ingredients. Exemplary suitable active ingredients include, among others, U.S.F.D.A. approved drugs for parenteral or oral dosage forms. Other active ingredients that can be used in cotherapy or adjunctive therapy with MG53 include, by way of example and without limitation, Benzoyl Peroxide, Beta-Hydroxy Acid (BHA), Dihydroxyacetone (DHA), Elastin, Sodium Hyaluronate, Zinc Oxide, Titanium Oxide, Vitamin E, or combinations thereof.

The therapeutically acceptable dose, maximum tolerated dose (MTD), and minimally effective dose (MED) for each of said active ingredients is well known and set forth in the respective U.S.F.D.A. approved product package insert for each said active ingredients.

A composition, dosage form or formulation of the invention can include one, two or more active ingredients in combination with MG53. The dose of each said active ingredient in said composition, dosage form or formulation of the invention will be a therapeutically effective dose including and above the MED and including and below the MTD.

In some embodiments, the combination treatment of MG53 with another active ingredient provides at least additive therapeutic efficacy. In some embodiments, said combination provides synergistic therapeutic efficacy. In some embodiments, MG53 reduces the occurrence of, reduces the level of, or eliminates adverse events caused by the other active ingredient.

The acceptable concentrations of said excipients are well known in the art and specific concentrations (amounts) thereof are set forth in the package insert or package label of known commercial products containing the same.

It should be understood that compounds used in the art of pharmaceutics may serve a variety of functions or purposes. Thus, if a compound named herein is mentioned only once or is used to define more than one term herein, its purpose or function should not be construed as being limited solely to that named purpose(s) or function(s).

In the examples below, ranges are specified for the amount of each ingredient. Ranges including “0” as the lowest value indicate an optional ingredient. The lower limit “>0” indicates the respective material is present.

As used herein, the terms “about” or “approximately” are taken to mean a variation or standard deviation of 10%, +5%, or ±1% of a specified value. For example, about 20 mg is taken to mean 20 mg±10%, which is equivalent to 18-22 mg.

As used herein, the term “prodrug” is taken to mean a compound that, after administration, is converted within a subject's body, e.g. by metabolism, hydrolysis, or biodegradation, into a pharmacologically active drug. The prodrug may be pharmacologically active or inactive. For example, a prodrug of MG53 (native or mutant) would be converted to the native form or mutant form, respectively, of MG53. The term “precursor” may also be used instead of the term “prodrug”.

As used herein, the term “derivative” is taken to mean: a) a chemical substance that is related structurally to a first chemical substance and theoretically derivable from it; b) a compound that is formed from a similar first compound or a compound that can be imagined to arise from another first compound, if one atom of the first compound is replaced with another atom or group of atoms; c) a compound derived or obtained from a parent compound and containing essential elements of the parent compound; or d) a chemical compound that may be produced from first compound of similar structure in one or more steps. For example, a derivative may include a deuterated form, oxidized form, dehydrated, unsaturated, polymer conjugated or glycosilated form thereof or may include an ester, amide, lactone, homolog, ether, thioether, cyano, amino, alkylamino, sulfhydryl, heterocyclic, heterocyclic ring-fused, polymerized, pegylated, benzylidenyl, triazolyl, piperazinyl or deuterated form thereof.

In the examples below, ranges are specified for the amount of each ingredient. Ranges including “0” as the lowest value indicate an optional ingredient. Compositions with quantities of ingredients falling within the compositional ranges specified herein were made. Compositions of the invention comprising quantities of ingredients falling within the compositional ranges specified herein operate as intended and as claimed.

In view of the above description and the examples below, one of ordinary skill in the art will be able to practice the invention as claimed without undue experimentation. The foregoing will be better understood with reference to the following examples that detail certain procedures for the preparation and use of compositions according to the present invention. All references made to these examples are for the purposes of illustration. The following examples should not be considered exhaustive, but merely illustrative of only a few of the many embodiments contemplated by the present invention. The methods described herein can be followed to prepare and use compositions of the invention and to practice methods of the invention.

MG53 was kindly provided by TRIM-edicine, Inc. (1275 Kinnear RD, Columbus Ohio 43212-1155).

### Example 1

### Preparation of AHPPE Intermediate

4-(hydroxymethyl)-phenylboronic acid pinacol ester (HPPE) (11.7 g, 50 mmol) was dissolved in dehydrated dichloromethane (DCM, 60 mL) in a flask, followed by adding TEA (6.1 g, 60 mmol). Then the flask was placed on ice to be pre-cooled, and after cooling to 0° C., acryloyl chloride (5.4 g, 60 mmol) in 5.4 mL anhydrous DCM was added dropwise for 1 h. Then the ice under the flask was removed and the reaction mixture was warmed back to room temperature with continuous stirring. After 10 h reaction, the products were filtered, washed by DI water and brine for 3 times, respectively. Then the products were concentrated using rotary evaporator and dried with MgSO₄. The monomer AHPPE was then obtained as faintly yellow powder and the yield was ˜90%.

### Example 2

### Preparation of AAHPPE Gel

Starting with the AHPPE of Example 1, the gel was prepared as follows.

Poly (NIPAAm-co-HEMA-co-AAcPB) was polymerized by dissolving dehydrated NIPAAM, HEMA and AHPPE in dioxane with 20 min purging of N₂. BPO was added as initiator, followed by another 20 min purging with N₂. The mixture was then placed in a preheated oil bath at 65° C. for 20 hours with continuous stirring. The products were obtained and purified by three times precipitation-redissolution cycle using tetrahydrofuran (THF)/hexane and THF/ethyl ether. The final products were further dried by vacuum oven and lyophilizer. Three different compositions of copolymers AHPPE-10, AHPPE-8 and AIPPE-6 were synthesized with the ratios of 75/15/10, 77/15/8 and 79/15/6 for NIPAAm/IEMA/AHPPE, respectively.

### Example 3

### AAHPPE Gel Solidification

Starting with the AAHPPE gel of Example 2, the gel (hydrogel solution) was prepared as follows. The purified copolymer was dissolved in DPBS, with continuous stirring at 4° C. overnight. 200 μL of hydrogel solution was transferred in a 1.5 mL microcentrifuge tube. The tubes were then placed in a 37° C. water bath and the gel solution solidified within seconds.

### Example 4

### Dermal Dressing Comprising AAHPPE Gel Alone

The AAHPPE gel of Example 2 was applied directly to the skin. Before application, the gel was fluid with a viscosity of less than about 0.1 Pa sec. An applicator was loaded with the fluid gel and placed on the skin of a subject, whereby the gel solidified within about 1 min to form a dressing.

### Example 5

### Dermal Dressing Comprising AAHPPE Gel and MG53

The AAHPPE gel (2 mL, 8 wt. % in aqueous vehicle, 160 mg) of Example 2 was mixed with MG53(2 mg, at a concentration of 1 mg/mL). rhMG53 peptide was dissolved in sterilized DPBS and further sterilized by filtering through 0.22 μm syringe filter for two times. The concentration of hydrogel solution was 8 wt. % and the rhMG53 solution in DPBS was thoroughly mixed with hydrogel solution at 4° C. overnight. The final concentration of rhMG53 was 1 mg/mL. The mixture was then applied directly to the skin, whereby the gel solidified within about 1 min to form a dressing. The gel provided a continuous release of MG53. Greater than about 60% wt of the total amount of MG53 was released in 21 days.

### Example 6

### AAHPPE Gel with Another Active Ingredient

The AAHPPE gel of Example 2 is mixed with another active ingredient.

The mixture is then applied directly to the skin, whereby the gel solidifies in less than five min to form a dressing. The gel provides a continuous release of the active ingredient.

### Example 7

### Evaluation of Antioxidant Activity of AAHPPE Gel

The ability of the AAHPPE gel (8 wt. % in DPBS) of Example 2 to sequester or capture reactive oxygen species was evaluated in Dulbecco's phosphate buffered saline (DPBS) in the presence of hydrogen peroxide (50 mM). Three different embodiments of the gel (AAHPPE-10, AAHPPE-8, AAHPPE-6) were evaluated. The loss of weight of gel corresponded to the extent of hydrogen peroxide sequestered.

AAHPPE-10, AAHPPE-8 and AAHPPE-6 (copolymer content of 8 wt. % in each) hydrogel solutions (200 μL) were transferred to 1.5 mL microcentrifuge tubes (tubes were weighed before use as w4), sitting in a 37° C. water bath for 1 h to complete gelation process. After gelation, the supernatants were removed from each tube and 200 μL of DPBS without H₂O₂ and with 50 mM H₂O₂ was added to the tubes. The sample tubes were taken out at each time point on week-0, week-1, week-2, week-3 and week-4. At each time point, the tubes were weighed after removing supernatants and recorded as w5. Finally, the samples were washed by DI H₂O and solutions were evaporated to dryness using lyophilizer and tubes weighed as w6. During the degradation, the medium (DPBS with/without H₂O₂) was exchanged every other day to guarantee the fresh H₂O₂ for use. Four repeats of samples were used for each condition (n=4). Additionally, the one-week degradation products were obtained, freeze dried and characterized using ¹H NMR using DMSO as the solvent. The copolymers AAHPPE6/8/10 before degradation were also dissolved in DMSO for NMR test at the same time.

### Example 8

Evaluation of Wound Healing Activity of AAHPPE gel with and without MG53

The ability of the AAHPPE gel (8 wt. % in DPBS, 200 μL for each wound) of Examples 2 and 5 to accelerate healing of a puncture wound was evaluated as follows.

Before wound incision, the mice hair on the back was shaved using an electric clipper. Betadine solution and alcohol prep pads were used to wipe the naked area on the back to avoid infection. Two wounds on left and right were created on each mouse using sterilized 5 mm-diameter biopsy punches (Integra Miltex, Plainboro, N.J.) so that the underlying dorsolateral skeletal muscle can be exposed. Wounds left open with no dressing on db/+ mice (C57BL/6 mice) and db/db mice were considered as control groups. 200 μL of hydrogel solution was injected on the wound subcutaneously.

### Example 9

### Evaluation of Release of MG53 from AAHPPE Gel

AAHPPE copolymer grades containing different monomer ratios (75/15/10, 77/15/8 and 79/15/6 for NIPAAm/HEMA/AHPPE) were prepared according to Example 1. The corresponding gels (AAHPPE-10, monomer ratio 75/15/10; AAHPPE-8, monomer ratio 77/15/8) were prepared by placing the copolymers in Dulbecco's Phosphate-Buffered Saline (DPBS) (copolymer concentration: 8 wt. %, 200 μL, 16 mg). The copolymers were solidified with slight heat and the release of MG53 over a period of 21 days was monitored by drawings aliquots and analyzing the supernatant. The concentration of released rhMG53 was measured by the microplate reader (Molecular Device). 280 nm wavelength was used in the absorption read mode. The standard curve was acquired by measuring absorbance of various concentrations of rhMG53 in DPBS (0, 0.25, 0.5, 0.75, 1.0, 1.25, 1.5, 1.75 mg/mL) using the plate reader. The observed standard curve was well fitted by a linear regression model. Therefore, the cumulative released rhMG53 concentration was calculated by dividing the release rhMG53 by the total rhMG53 encapsulated in the hydrogel.

| | Content of MG53 mg/ml of gel | |
|---|---|---|
| Name | AAHPPE-10 | AAHPPE-8 |
| 9A | 1 | 0 |
| 9B | 0.5 | 0 |
| 9C | 0 | 1 |
| 9D | 0 | 2 |

### Example 10

### Comparison of Antioxidant Activity of AAHPPE Gel and APLA Gel

APLA (poly (NIPAAm-co-HEMA-co-APLA)) is a non-reactive gel, meaning it is non-responsive to reactive oxygen species (ROS).

Fenton reaction method. 200 μL of APLA or AHPPE hydrogel solution was mixed with 200 μL FeSO₄ solution (2 mM), 200 μL Safranin 0 (360 μg/mL) and 320 μL H₂O₂(6 wt. %) solution, was incubated for 10 min, followed by heating in a 55° C. water bath for 30 min. 200 μL of DI H₂O was used as blank group. For the control group, DI H₂O was used instead of AHPPE gel sample and H₂O₂ solution. The mixtures were then cooled to room temperature and the absorption was read at 492 nm using a plate reader (Molecular Device). The scavenging capacity of hydroxyl radicals was determined by the equation (1):

$$Hydroxyl radical scavenging effect ( \% ) = \frac{{A}_{sample} - {A}_{blank}}{{A}_{control} - {A}_{blank}} \times 100 \qquad ( 1 )$$

Pyrogallol assay method. 500 μL of Tris-HCl (50 mM, pH=8.1) was added into a 200 μL gel solution, followed by the drop-wise addition of 80 μL pyrogallol (3 mM) in the dark. At last, 20 μL HCl (8 M) solution was added into the mixture to stop the reaction. For the control group, DI H₂O was used to substitute the gel solution. The absorption was measured using the plate reader. The capability to scavenge superoxide was then calculated based on the equation (2):

$$Superoxide scavenging effect ( \% ) = \frac{{A}_{control} - {A}_{sample}}{{A}_{control}} \times 100 \qquad ( 2 )$$

### Example 11

### Evaluation of Hair Growth Promoting Activity of AAHPPE Gel with and without MG53

The ability of the AAHPPE gel (8 wt. % in DPBS, 200 μL, 16 mg of copolymer) of Examples 2 and 5 to promote hair growth wound was evaluated as follows. A digital camera (Panasonic, Osaka, Japan), a metric ruler and a digital caliper (CD-6″ CSX, Japan) were used for post-surgery wound measurements at each time point (day 0, 2, 4, 6, 8, 10, 12, 14, 16, 18, 20 and 22 post-surgery). The camera was used to capture the wound along with the ruler inside the field. Image-J was then used to accurately calculate wound size. The area of each wound was recorded.

### Example 12

### Evaluation of AAHPPE Gel with and without MG53 for Protection Against Oxidative Stress

The ability of the AAHPPE gel of Examples 2 and 5 to protect tissue against oxidative stress was evaluated as follows. FSCs survival on 2D hydrogels with and without H₂O₂ was monitored for 5 days. MTT assay and live cell images illustrated that ROS responsive AHPPE gel enhanced FSCs survival rate under oxidative stress compared with non-ROS responsive APLA gel.

Method A: (AHPPE gel and APLA gel without MG53): FSCs used for in vitro experiments were passage 4-7. The culture medium used was the complete media with Serum and 1% penicillin. FSCs were digested using trypsin-EDTA (0.25%) from T-75 culture plates and re-suspended in pre-warmed culture medium without serum. The 96 well plate was pre-coated evenly with 200 μL of the hydrogel solution (8 wt. % AHPPE gel and 8 wt. % APLA gel was used as a control) and balanced in a 37° C., 5% CO₂ and 21% 02 incubator. 200 μL of FSCs suspension were seeded into the well with a density of 10,000 cells/mL. The plate was incubated in 37° C. for 24 h and then the culture medium was replaced with fresh medium (500 μM H₂O₂ or without H₂O₂). The medium was exchanged every other day to guarantee fresh H₂O₂.Three plates were used for different time points at day 1, 3 and 5. At each time point, 20 μL of MTT solution (3-(4,5-dimethylthiazol-2-yl)-2,5-diphenyltetrazolium bromide, 5 mg/mL) was added to the well and incubated for another 4 h before adding 200 μL DMSO. The intensity of fluorescence was read at 560 nm and background at 670 nm was subtracted. The groups included AHPPE gel without H₂O₂, APLA gel without H₂O₂, AHPPE gel with 500 μM H₂O₂ and APLA gel with 500 μM H₂O₂. In addition, FSCs were stained with CMDil (a fluorescent dye used as live cell tracker, Thermofisher) before seeding on the hydrogel. At each time point, the samples were taken out and the medium were removed. Live cell images were obtained by confocal microscope using Z-stack mode.

Method B: (FSCs with or without MG53): FSCs were cultured in 96-well plate with a seeding density of 2,000 cells/well. FSCs were treated with 500 μM H₂O₂ 24 h after seeding and the groups included without MG53, with 1 μg/mL MG53 and 10 μg/mL MG53. MTT assay was performed 5 days post treatment to evaluate the cell viability with or without different concentrations of MG53. TUNEL assay was also performed using TUNEL assay kit-HRP (abcam) to validate the effect of MG53 on reducing cell apoptosis. FSCs were cultured on glass slide inserted in a 6-well plate and treated with or without MG53/500 μM H₂O₂ for 24 h. FSCs were then washed by cold DPBS twice and resuspended in 4% formaldehyde in DPBS at a density of 1×10⁶/mL, and incubated at room temperature for 10 min. The cells were then rehydrated in TBS, specimen permeabilized using proteinase K with 1/100 dilution in dH₂O. Inactivation of endogenous peroxidases was achieved by adding diluted H₂O₂ in methanol (1/10), followed by labeling reaction using TdT labeling reaction mixture, and development using methyl green counterstain solution provided by the kit.

### Example 13

### MG53-Containing Cream

Lyophilized MG53 is mixed with cream base (1.5 to 10 mg MG53 per oz of cream base) until homogeneity is reached. The cream base comprises water (about 65-75%% w/w), at least one preservative (about 0.1-1% w/w), at least one humectant (about 3-5% w/w), at least one exfoliant about 1-5% w/w), at least one penetration enhancer (about 5-15% w/w), at least one emollient (about 1-7% w/w), at least one protectant (skin conditioning agent; about 0.1-5% w/w), at least one antimicrobial (about 0.1-5% w/w), at least one zinc salt (about 0.1-1% w/w).

### Example 14

### Oral Dosage Form of rhMG53 and EUDRAGIT S-100

rhMG53 is provided by TRIM-edicine, Inc. (Columbus, Ohio). EUDRAGIT S-100 (Poly(methacylic acid-co-methyl methacrylate) 1:2) is provided by EVONIK healthcare.evonik.com/product/health-care/en/. The following procedure is used to prepared beads.

In a 100 mL beaker, add 35 mL water, and stir. While stirring add Eudragit S-100 powder (1.4 g), then and 12N NH₄OH (0.82 mL).

Add 2-hydroxypropyl)-β-cyclodextrin (0.24 g) to a 10 mL water (CD: 24 mg/mL).

Prepare a solution of MG53 (70 mg in ˜15.5 mL PBS) at a pH 8. To this solution, add 10 mL of the CD solution and 10 mL of water for a total volume of 35.55 mL.

Mix the MG53/CD solution with the Eudragit solution while stirring.

Spray dry the resulting suspension to form the powdered dosage form containing MG53 (70 mg), EUDRAGIT (1.4 g), salts (130 mg), and CD (0.24 g) for a total solids content of 1.77 g or a MG53-loading of 40 mg/g of solid (4% loading). Spray drying conditions used: nozzle size—0.6 mm; air speed—0.3 m³/min; air outlet temp: 38 C; room temperature: 24 C; room humidity: 53%.

The powder can be included in a capsule, caplet, tablet or other oral dosage form.

### Example 15

### Quantitation of Hair Follicles

Skin samples dissected from the back of experimental animals were fixed in 10% formalin, then processed and embedded in 4 m thick paraffin sections. skin sections were stained with hematoxylin-eosin (H&E). Quantification of the number of hair follicles was made by count hair follicle numbers in each 1000 μM skin section.

### Example 16

### Evaluation of MG53 Upon Hair Growth in Mice

Dorsal hair was shaved using electric clippers and cleared by Nair™ (hair remover lotion). Then the mice were immediately received a subcutaneous injection of either 200 μl saline (as control) or rhMG53 (2 mg/kg), and treated daily for 7 successive days. The images were captured at indicated time points by a digital camera (Panasonic DMC-ZS3, Japan).

### Example 17

### Evaluation of MG53 Upon Hair Growth in Rats

Dorsal hair was shaved using electric clippers. Then the rats were immediately received a subcutaneous injection of either BSA (1 mg/kg) or rhMG53 (1 mg/kg), and treated daily for 7 successive days. The images were captured at indicated time points by a digital camera (Panasonic DMC-ZS3, Japan).

Dorsal hair was shaved using electric clippers and disinfected. Two full thickness dermal wounds were created using sterile ϕ 6 mm biopsy punches (Integra™ Miltex®). And the rats were immediately received a subcutaneous injection of either 200 μl saline (as control) or rhMG53 (1 mg/kg) and treated daily for 7 successive days. Skin samples were stained with Hematoxylin-Eosin (H&E). Quantification of the number of hair follicles revealed improved hair follicle growth in the rats with rhMG53 treatment compared to saline control at day 14 following wound.

### Example 18

### Evaluation of MG53 Upon Improvement of Survivability of Human Hair Follicle Stem Cells Following Treatment with Hydrogen Peroxide

FSCs were cultured in 96-well plate with a seeding density of 2,000 cells/well. FSCs were treated with 500 μM H₂O₂ 24 h after seeding and the groups included without MG53, with 1 μg/mL MG53 and 10 μg/mL MG53. FSCs were then washed by cold DPBS twice and resuspended in 4% formaldehyde in DPBS at a density of 1×10⁶/mL, and incubated at room temperature for 10 min. The specimens were then rehydrated in TBS, and permeabilized using proteinase K with 1/100 dilution in dH2O. Inactivation of endogenous peroxidases was achieved by adding diluted H2O2 in methanol (1/10), followed by labeling reaction using TdT labeling reaction mixture, and development using methyl green counterstain solution provided by the kit. All staining was imaged using a KEYENCE microscope. Apoptotic cells were quantified from the acquired images.

### Example 19

### Evaluation of MG53-Containing Cream for Improvement of Hair Growth in Diabetic Mice

A mouse model of chronic excisional dermal wound (4 mm×4 mm Biopsy punch) was applied on the db/db mice. Topical creams containing 10 mg/oz of rhMG53 or control (without rhMG53) were applied every other day. At the day 12, the wound tissue was excised, fixed, embedded. H/E stating was performed to evaluate the hair follicle numbers in wound area.

### Example 20

### Evaluation of MG53-Containing Cream for Treatment of Skin after Laser Ablation

On the day of the laser ablation procedure, patients had their photo taken for comparative data. Immediately following the treatment, a marketed conventional moisturizer product was applied to the right side of the patient's face and MG53 cream was applied to the patient's left side of the face. The creams were applied daily for 7 days post-procedure. The picture was taken daily. The figure was shown for the day 4.

### Example 21

Evaluation of MG53-containing cream for treatment of red blemishes on skin

An elderly white male diagnosed with Bullous pemphygoid-like skin condition. Similar to butterfly skin syndrome. Skin is fragile and breaks easily with slight mechanical stress. Skin also blisters easily before bursting, exposing open wounds that heal slowly due to skin condition. Individual frequently tears skin in his sleep waking up with new tears/wounds and bloodied sheets/clothes. MG53 cream was applied daily to both new and scabbed wounds. MG53 cream application has significantly reduced time to healing by at least 1 week. This rate of healing was more rapid with daily application AND coverage with bandage. Therefore, keeping the wound moist with MG53 and covered allowed faster epithelialization of the wounds. Furthermore, areas where it is applied regularly (i.e. wrist) are less prone to tearing and blistering.

### Example 22

### Evaluation of MG53-Containing Cream for Reduction of Skin Wrinkles

On the day of the CO₂ procedure, patients had their photo taken for comparative data. Immediately following the treatment, a conventional commercial cream product was applied to the left side of the patient's face and MG53 cream was applied to the patient's right side of the face. The creams were applied daily for 28 days post-procedure. The picture was taken daily. The figure was shown for week 3 post-procedure.

### Example 23

### Evaluation of MG53-Containing Cream for Treatment of Chronic Open Wound in Elderly Person

An elderly male diagnosed with Bullous pemphygoid-like skin condition was in otherwise good health. Skin was fragile and broke easily with slight mechanical stress. The hand wound was treated with MG53-containing cream daily for 3 weeks. The skin became more resistant to tearing by slight mechanical stress.

### Example 24

### Evaluation of MG53-Containing Cream for Treatment of Cryogenically Treated Tissue

An elderly male had a mole removed from the face by cryofreeze. The dermatologist stated that at least 1 week would be needed before pain would subside and 2 weeks before visible improvements in skin healing would be observed. Daily application of MG53-containing cream resulted in pain reductions after 1-2 days and rapid healing within 1 week. No bandage was applied. Cream was applied 2-3 times per day.

### Example 25

### rhMG53 Protein Production and Quality Control

The following process was used to produce native MG53 protein.

E. coli fermentation was used to obtain high quality (>97% purity) rhMG53 (recombinant human MG53) protein as described by Zhu et al. (“Polymerase transcriptase release factor (PTRF) anchors MG53 protein to cell injury site for initiation of membrane repair” in The Journal of biological chemistry (2011), 286, 12820-12824) and Weisleder et al. (Recombinant MG53 protein modulates therapeutic cell membrane repair in treatment of muscular dystrophy. Science translational medicine (2012), 4, 139ra185), the entire disclosures of which are hereby incorporated by reference. The membrane protective activity of rhMG53 from each preparation was determined with established micro-glass bead injury assay as described previously (ibid).

### Example 26

### Treatment of Wrinkles and Melanin Production with rhMG53-Containing Mask

A MG53-containing mask was prepared by saturating a commercially available disposable dry mask cloth with a normal saline solution containing rhMG53 (20 microg/mL).

About two to three times per week, an MG53-containing mask was placed on the face of a subject after the skin had been cleaned. The mask was administered at night for about 10-30 min per treatment. The subject was administered the mask for about two to three weeks or more. The melanin content and number of wrinkles were quantified with a skin Cutometer before and after the two-week treatment period. Results are depicted in FIGS. 11A-11B.

All data are expressed as mean±S.D. Groups were compared by Student's t test and analysis of variance for repeated measures. A value of p<0.05 was considered significant.

For any range herein, the upper and lower limits thereof are considered as being part of the range. Moreover, all integer and fractional values within said ranges are also considered as being within said range. Accordingly, all integers and fractional values within each specified range are hereby incorporated by reference.

All values disclosed herein may have standard technical measure error (standard deviation) of ±10%. The term “about” or “approximately” is intended to mean±10%, ±5%, ±2.5% or ±1% relative to a specified value, i.e. “about” 20% means 20±2%, 20±1%, 20±0.5% or 20±0.25%. The term “majority” or “major portion” is intended to mean more than half, when used in the context of two portions, or more than one-third, when used in the context of three portions. The term “minority” or “minor portion” is intended to mean less than half, when used in the context of two portions, or less than one-third, when used in the context of three portions. It should be noted that, unless otherwise specified, values herein concerning pharmacokinetic or dissolution parameters are typically representative of the mean or median values obtained.

The above is a detailed description of particular embodiments of the invention. It will be appreciated that, although specific embodiments of the invention have been described herein for purposes of illustration, various modifications may be made without departing from the spirit and scope of the invention. Accordingly, the invention is not limited except as by the appended claims. All of the embodiments disclosed and claimed herein can be made and executed without undue experimentation in light of the present disclosure.

1) A cosmeceutical composition comprising MG53 and one or more cosmetically acceptable excipients.

2) The cosmeceutical composition of claim 1 further comprising a) one or more antioxidants; b) one or more zinc salts; c) aqueous liquid vehicle; or d) a combination thereof.

3) The cosmeceutical composition of claim 2, wherein a) the molar ratio of MG53 to antioxidant ranges from 0.01:1 to 10:1; b) the molar ratio of MG53 to zinc salt is about 1:2 or more; and/or c) the content of aqueous liquid vehicle is at least 30% wt.

4) The cosmeceutical composition of claim 1, wherein said composition a) is adapted for topical administration, dermal administration, transdermal administration, or subcutaneous administration; and/or b) is a gel, cream, or ointment.

5) The cosmeceutical composition of claim 1, wherein said composition a) is a gel comprising at least one cosmetically acceptable antioxidant polymer, an effective amount of MG53, and at least one liquid vehicle, wherein said gel exhibits a low viscosity at less than about 15° C., and said gel solidifies or semi-solidifies (thicken substantially into a high viscosity mass) after contact with skin, or after contact with a surface of at least about 18° C., at least about 20° C., or at least about 25° C.; b) is a cream comprising a cream base and an effective amount of MG53; c) is a cosmeceutical ointment comprising an ointment base and an effective amount of MG53; or d) is a hydrogel that is a liquid prior to topical application to skin and becomes a gel, semi-solid or solid after topical application to skin.

6) The cosmeceutical composition of claim 5, wherein a) said cream base comprises one or more cosmetically acceptable (and/or pharmaceutically acceptable) excipients; b) said antioxidant polymer comprises ROS-scavenging copolymer; c) said hydrogel comprises boronate ester-based copolymer, said hydrogel exhibiting lower viscosity at about a lower temperature and higher viscosity at a higher temperature; and/or d) said hydrogel i) exhibits lower viscosity at about 4° C. and higher viscosity at about 37° C.; ii) is a liquid at about 4° C. and a gel, semi-solid or solid at about 37° C.; and/or iii) at about 4° C. is flowable by hand through an 18-30 gauge needle engaged to a syringe and at about 37° C. is not flowable by hand through an 18-30 gauge needle engaged to a syringe; e)

7) The cosmeceutical composition of claim 6, wherein said copolymer a) is a terpolymer comprising one or more acrylamide monomer(s), one or more methacrylate monomer(s), and one or more boronate ester acrylate monomer(s); and/or b) excludes an alkyne group-containing monomer.

8) The cosmeceutical composition of claim 7, wherein a) said one or more acrylamide (AM) monomer(s) refers to (N-alkyl) acrylamide (NAAM) or homolog(s) or analog(s) thereof; b) said one or more methacrylate (MAc) monomer(s) refers to (hydroxyalkyl) methacrylate (HAMc) or homolog(s) or analog(s) thereof; c) said one or more boronate ester acrylate (BEAc) monomer(s) refers to 4-(hydroxyalkyl)-phenylboronic acid, pinacol ester) acrylate (HPPE-Ac) or homolog(s) or analog(s) thereof, or d) a combination thereof.

9) The cosmeceutical composition of claim 8, wherein a) said one or more acrylamide (AM) monomer(s) or homolog(s) or analog(s) is defined by the following chemical structure 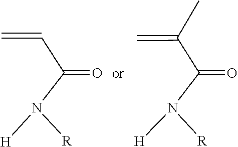
wherein R is propyl, isopropyl, butyl, isobutyl or other alkyl group of 5 or less carbon atoms in length;
b) said one or more methacrylate (MAc) monomer(s) or homolog(s) or analog(s) is defined by the following chemical structure 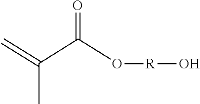
wherein R is (CH₂)ₙ; and n=2, 3, or 4; and/or
c) said one or more boronate ester acrylate ester (BEAc) monomer(s) or homolog(s) or analog(s) is defined by the following chemical structure

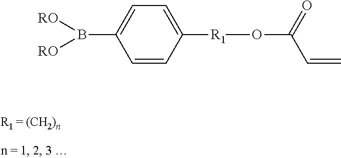

10) The cosmeceutical composition of claim 9, wherein alkyl is independently selected upon each occurrence from the group consisting of (C₁-C₂)-alkyl or (C₃-C₈)-alkyl, which alkyl may be linear, branched, or cyclic.

11) The cosmeceutical composition of claim 6, wherein said terpolymer is defined by the formula
poly((AM)ₘ-co-(MAc)ₙ-co-(BEAc)ₓ), a)
wherein: m is in the range of about 75 to about 85, n is in the range of about 5 to about 20, and x is in the range of about 5 to about 20;
poly((NAAM)ₘ-co-(HAMAc)ₙ-co-(HPPE-Ac)ₓ) b)
wherein: m is in the range of about 75 to about 85, n is in the range of about 5 to about 20, and x is in the range of about 5 to about 20; or
poly((NIPAA)ₘ-co-(HEMA)ₙ-co-(AIPPE)x), c)
wherein NIPAAM is N-isopropylacrylamide, HEMA is hydroxyethyl methacrylate, AHPPE is (4-(hydroxymethyl)-phenylboronic acid, pinacol ester) acrylate (or 4-(4,4,5,5-tetramethyl-1,3,2-dioxaborolan-2-yl)phenyl]methyl ester).

12) A method of improving the appearance of skin by administering to the skin a cosmetically effective amount of the cosmeceutical composition of claim 1.

13) The method of claim 12, wherein said improving is selected from the group consisting of reducing wrinkles, reducing melanin deposition, reducing irritation, reducing inflammation, reducing redness, reducing scarring, reducing collagen formation, reducing differentiation of stem cells into fibroblasts, reducing the period of time for amelioration of dermal of epidermal condition(s).

14) A method of preventing reactive oxygen species (ROS)-related oxidative damage to skin, said method comprising prophylactically administering to the skin a cosmetically effective amount of the cosmeceutical composition of claim 1 prior to exposure of said skin to the ROS.

15) A method of sequestering, neutralizing or eliminating reactive oxygen species (ROS) in skin comprising administering to the skin a cosmetically effective amount of the cosmeceutical composition of claim 1 after exposure of said skin to the ROS.

16) A method of improving hair growth, the method comprising administering to the skin of a subject in need thereof a cosmetically effective amount of the cosmeceutical composition of claim 1.

17) The method of claim 16, wherein said improving hair growth includes at least one of increasing hair strand thickness, improving hair follicle health, increasing the number of hair follicles per unit area of skin, or increasing rate of hair growth.

18) A method of removing skin blemishes or improving the appearance of skin blemishes, the method comprising administering to the skin of a subject in need thereof a cosmetically effective amount of the cosmeceutical composition of claim 1, wherein said removing or improving the appearance of skin blemishes comprises reducing the appearance or number of wrinkle(s), reducing the appearance or number of blotch(es), reducing melanin deposition, and/or reducing the appearance or number of reddened patch(es).

19) A method of healing injured tissue, the method comprising administering to injured tissue an effective amount of the cosmeceutical composition of claim 1, wherein the skin has not been physically injured, such as by impact force, burning, irradiation, or cutting, and/or the skin is otherwise healthy except for exhibiting cosmetically undesired features.

20) Method of treating a healed wound comprising administering to said healed wound, the cosmeceutical composition of claim 1, whereby said administering results in reduction of collagen formation at the site of said healed wound, and/or increase of follicle density at the site of said healed wound.
